(19) United States
(12) Patent Application Publication
(10) Pub. No.: US 2004/0042997 A1
(43) Pub. Date: Mar. 4, 2004

(54) Method of regenerating human tissue

(76) Inventor: Donnie Rudd, Sugar Land, TX (US)

Correspondence Address: LADAS & PARRY, 224 SOUTH MICHIGAN AVENUE, SUITE 1200, CHICAGO, IL, 60604, US

(21) Appl. No.: 10/233,342

(22) Filed: Sep. 3, 2002

(51) Int. Cl. A61K 38/19

(52) U.S. Cl. 424/85.1

(57) ABSTRACT

A method for regenerating a human individual's tissue is disclosed comprising determining the white blood cell content of the individual, administering granulocyte colony-stimulating factor to the individual while monitoring the white blood cell content of the individual, continuing the administration of the granulocyte colony-stimulating factor to the individual until the white blood cell content is more than twice its original amount, maintaining the administration of the granulocyte colony-stimulating factor to the individual at a level that maintains the white blood cell content at least at twice its original amount, monitoring the individual's tissue to be regenerated, and discontinuing the administration of the granulocyte colony-stimulating factor to the individual when the tissue regenerates. Also disclosed is the above method which includes removing blood cells from the individual, controllably expanding the blood cells while maintaining their three-dimensional geometry and their cell-to-cell geometry and reintroducing the blood cells into the individual while administering the granulocyte colony-stimulating factor.

### CROSSREFERENCES TO RELATED APPLICATIONS

Not applicable.

### STATEMENT REGARDING FEDERALLY SPONSORED RESEARCH

Not applicable.

### BACKGROUND OF THE INVENTION

The present invention relates to regenerating human tissue.

Regeneration of human tissue has long been a desire of the medical community. Thus far, repair of human tissue has been accomplished largely by transplantations of like tissue from a donor. Beginning essentially with the kidney transplant from one of the Herrick twins to the other and later made world famous by South African Doctor Christian Barnard's transplant of a heart from Denise Darval to Louis Washkansky on Dec. 3, 1967, tissue transplantation became a widely accepted method of extending life in terminal patients.

Transplantation of human tissue, from its first use, encountered major problems, primarily tissue rejection due to the body's natural immune system. This often caused the use of tissue transplantation to have a limited prolongation of life (Washkansky lived only 18 days past the surgery).

In order to overcome the problem of the body's immune system, numerous anti-rejection drugs (e.g. Inuran, Cyclosporine) were soon developed to suppress the immune system and thus prolong the use of the tissue prior to rejection. However, the rejection problem has continued creating the need for an alternative to tissue transplantation.

Bone marrow transplantation was also used, and is still the procedure of choice for treatment of some illnesses, such as leukemia, to repair certain tissues such as bone marrow, but bone marrow transplantation also has problems. It requires a match from a donor (found less than 50% of the time); it is painful, expensive, and risky. Consequently, an alternative to bone marrow transplantation is highly desirable. Transplantation of tissue stem cells such as the transplantation of liver stem cells found in U.S. Pat. No. 6,129,911 have similar limitations rendering their widespread use questionable.

In recent years, researchers have experimented with the use of pluripotent embryonic stem cells as an alternative to tissue transplant. The theory behind the use of embryonic stem cells has been that they can theoretically be utilized to regenerate virtually any tissue in the body. The use of embryonic stem cells for tissue regeneration, however, has also encountered problems. Among the more serious of these problems are that transplanted embryonic stem cells have limited controllability, they sometimes grow into tumors, and the human embryonic stem cells that are available for research would be rejected by a patient's immune system (Nature, Jun. 17, 2002). Further, widespread use of embryonic stem cells is so burdened with ethical, moral, and political concerns that its widespread use remains questionable.

Certain human cells have been shown to be regenerated. For instance, U.S. Pat. No. 6,261,549 provides a method for recovering an isolated, culture-expanded population of human mesenchymal stem cells from the mesenchymal stem cell-enriched peripheral blood of an individual. U.S. Pat. No. 6,383,480 discloses use of the MK family that is used alone as an agent for proliferating hematopoictic stem cells and hematopoietic precursor cells. U.S. Pat. No. 6,162,427 discloses the use of G-CSF in combination with a chemotherapeutic agent (in particular, cyclophosphamide) to produce a pharmaceutical preparation for boosting the mobilization of hematopoietic stem cells from bone marrow. While each of these patents and the references disclosed therein utilize granulocyte colony-stimulating factor to achieve some type of cell growth, they do not provide a method for effecting human tissue repair by utilizing an increased blood cell count to increase the hematopoietic cells to an extent that allows the body's own mechanisms to repair the damaged tissue.

It can therefore be seen that a need exists to provide a method of human tissue repair not based on organ transplantation or embryonic stem cell utilization.

### SUMMARY OF THE INVENTION

The present invention is a method of regenerating a human individual's tissue. The method comprises determining the white blood cell content of the individual, administering granulocyte colony-stimulating factor to the individual while monitoring the white blood cell content of the individual, continuing the administration of the granulocyte colony-stimulating factor to the individual until the white blood cell content is more than twice its original amount, maintaining the administration of the granulocyte colony-stimulating factor to the individual at a level that maintains the white blood cell content at least at twice its original amount, monitoring the individual's tissue to be regenerated, and discontinuing the administration of the granulocyte colony-stimulating factor to the individual when the tissue regenerates.

It is an object of this invention to provide a method for repairing human tissue.

It is a further object of this invention to use a combination of a blood cell stimulating factor along with an individuals expanded blood to increase the ability of the body of an individual to repair body tissue.

It is still another object of this invention to provide a method of repairing human tissue without the use of organ transplantation or embryonic stem cell use.

These and still other objects and advantages of the present invention will be apparent from the description of the preferred embodiments that follow. However, the claims should be looked to in order to judge the full scope of the invention.

### DETAILED DESCRIPTION OF THE INVENTION

This invention may be more fully described by the preferred embodiment as hereinafter described.

In the preferred embodiment of this invention blood cells are removed from a patient. A subpopulation of these cells is currently referred to as adult stem cells. The blood cells are placed in a bioreactor such as that described in U.S. Pat. No. 5,702,941. The bioreactor vessel is rotated at a speed that provides for suspension of the blood cells to maintain their three-dimensional geometry and their cell-to-cell support and geometry. During the time that the cells are in the reactor, they are fed nutrients and toxic materials are removed. A subpopulation of these cells is expanded creating a large amount of cells. The expansion must be at least seven times and preferably within seven days. The cells are then injected intravenously or directly into the tissue. Prior to the cells being injected into the body, the individual's white blood cell count is taken. Concurrent with the infusion of the injected cells, the individual is injected with 30 mcg of granulocyte colony-stimulating factor per kg of body weight. The injection of granulocyte colony-stimulating factor continues for at least seven days. During this time, the white blood cell count is monitored. The injections of granulocyte colony-stimulating factor are continued for seven days after the white blood cell count has doubled. The method can be used to repair liver tissue, hematopoietic tissue, blood vessels, skin tissue, muscle tissue, gut tissue, pancreatic tissue, central nervous system cells, bone, cartilage, connective tissue, pulmonary tissue, spleen tissue, and other body tissue.

In still another embodiment of this invention, peripheral blood (PB) cells are obtained from a person needing tissue repair. In brief, mononuclear cells (MNCs) are obtained from the first apheresis product collected from the donors. Prior to apheresis, the individual's white blood cell count is taken. Concurrent with the infusion of the injected cells, the individual is injected with 30 mcg of granulocyte colony-stimulating factor per kg of body weight. The injection of granulocyte colony-stimulating factor continues for at least seven days. During this time, the white blood cell count is monitored. The injections of granulocyte colony-stimulating factor are continued for seven days after the white blood cell count has doubled. MNCs are collected by subjecting the donor's total blood volume to 3 rounds of continuous-flow leukapheresis through a Cobe Spectra cell separator.

Collected MNCs (0.75×10⁶ cells/ml) are suspended in Iscove's modified Dulbecco's medium (IMDM) (GIBCO, Grand Island, N.Y.) supplemented with 20% either fetal calf serum (FCS) (Flow Laboratories, McClean, VA), 5% human albumin (HA) or 20% human plasma, and 100 ng/ml recombinant human stem cell factor (SCF) (Amgen). The culture mix is injected into 300 ml or 500 ml Life Cell nonpyrogenic plastic bags (Baxter, Deerfield, Ill.) and placed in a humidified incubator at 37EC under an atmosphere of 5% CO₂. The culture bags are inspected daily.

Hematopoietic colony-forming cells are assayed using a modification of a previously described assay. In brief, 10⁵ MNCs are cultured in 0.8% methylcellulose with IMDM, 30% FCS, 1.0 U/ml erythropoietin (Amgen), 50 ng/ml recombinant human GM-CSF (Immunex Corp., Seattle, Wash.), and 50 ng/ml SCF (Amgen). One-milliliter aliquots of each culture mixture are then placed in 35-mm Petri dishes (Nunc Inc., Naperville, Ill.) and incubated in duplicate at 37EC in air in a humidified atmosphere of 5% CO₂. All cultures are evaluated after 7 days for the number of burst-forming unit-erythroid (BFU-E) colonies (defined as aggregates of more than 500 hemoglobinized cells or 3 or more erythroid subcolonies), for the number of colony-forming units granulocyte-macrophage (CFU-GM) colonies of granulocytic or monocyte-macrophage cells or both, and for the number of CFU-granulocyte-erythroid-macrophage-megakaryocyte (CFU-GEMM) containing all elements. Individual colonies are plucked from the cultures with a micropipette and analyzed for cellular composition.

Lymphocytes are analyzed by 2-color staining using the following antibody combinations: CD56+CD16-PE/CD3-FITC, CD3-PE/CD4-FITC, CD3PE/CD8-FITC, CD19-PE. Controls include IgG1-PE/IgG1-FITC for isotype and CD14-PE/CD45-FITC for gating. Progenitor cells are analyzed by 3-color staining with the fluorochromes PerCP/PE/FITC using the following antibody combinations: CD45/CD90/CD34, CD45/CD34/CD38, CD45/CD34/CD33, and CD45/CD34/CD15. CD45/IgG1/IgG1 is used as a control. In brief, 10⁶ cells from the donor are incubated with 10:1 of antibodies at 2-8EC for 15 minutes in the dark and then washed twice in phosphate-buffered saline. Then the cells are resuspended, fixed with 1% formaldehyde, and analyzed on a FACScan flow cytometer (Becton-Dickinson) equipped with CELLQuest software (Becton Dickinson). For analyses of lymphocytes, 10,000 cells are acquired from each tube, and then gated on the basis of the forward and right angle light scatter patterns. The cutoff point is visually set at a level above background positivity exhibited by isotype controls. For analyses of progenitor cells, 75,000 cells from each tube is acquired and then sequentially gated.

Incubation of the donors' PB cells in this tissue culture system significantly increases the numbers of hematopoietic colony-forming cells. A constant increase in the numbers of CFU-GM (up to 7-fold) and CFU-GEMM (up to 9-fold) colony-forming cells is observed up to day 7 with no clear plateau.

Incubation of MNCs from normal donors in this tissue culture system significantly increases the numbers of CD34+ cells. The average number of CD34+ cells increased 10-fold by day 6 of culture and plateaus on that same day. The relative number of CD34+ cells co-expressing the myeloid-lineage markers CD15 and CD33 increases significantly by days 5 and 6. When the white blood cells have doubled, the cells are reinjected into the patient. The injection can be an injection of the cells into the bloodstream or, as I now prefer, an injection directly into the injured tissue such as the liver.

It must be understood that this invention in its basic claim is for the utilization of granulocyte colony-stimulating factor in the doubling of the white blood cells and that although a completely new and novel approach of including it with expanded blood cells is shown as the preferred embodiment, the use of expanded cells is not necessary for the basic invention.

Having fully described my invention, what I claim as my invention is:

1. A method for regenerating a human individual's tissue comprising determining the white blood cell content of the individual, administering granulocyte colony-stimulating factor to the individual while monitoring the white blood cell content of the individual, continuing the administration of the granulocyte colony-stimulating factor to the individual until the white blood cell content is more than twice its original amount, maintaining the administration of the granulocyte colony-stimulating factor to the individual at a level that maintains the white blood cell content at least at twice its original amount, monitoring the individual's tissue to be regenerated, and discontinuing the administration of the granulocyte colony-stimulating factor to the individual when the tissue regenerates.

2. A method as in claim 1 wherein the tissue to be regenerated is liver tissue.

3. A method as in claim 1 wherein the granulocyte colony-stimulating factor is administered in an amount of mcg/kg of body weight/day for at least seven days.

4. A method for regenerating a human individual's tissue comprising removing blood cells from the individual, controllably expanding the blood cells while maintaining their three-dimensional geometry and their cell-to-cell geometry, reintroducing the blood cells into the individual, determining the white blood cell content of the individual, administering granulocyte colony-stimulating factor to the individual while monitoring the white blood cell content of the individual, continuing the administration of the granulocyte colony-stimulating factor to the individual until the white blood cell content is more than twice its original amount, maintaining the administration of the granulocyte colony-stimulating factor to the individual at a level that maintains the white blood cell content at least at twice its original amount, monitoring the individual's tissue to be regenerated, and discontinuing the administration of the granulocyte colony-stimulating factor to the individual when the tissue regenerates.

5. A method as in claim 4 wherein the tissue to be regenerated is liver tissue.

6. A method as in claim 4 wherein the granulocyte colony-stimulating factor is administered in an amount of mcg/kg of body weight/day for at least seven days.
